(19) United States
(12) Patent Application Publication
(10) Pub. No.: US 2025/0060313 A1
(43) Pub. Date: Feb. 20, 2025

(54) MOLECULAR IMAGING METHOD AND SYSTEM OF RAMAN SPECTRA BASED ON MACHINE LEARNING CASCADE

(71) Applicant: Jinling Hospital, Affiliated Hospital of Medical School, Nanjing University, Jiangsu (CN)

(72) Inventors: Guangming Lu, Jiangsu (CN); Qingqing Zhou, Jiangsu (CN); Meng Chen, Jiangsu (CN); Jingxing Guo, Jiangsu (CN); Qiang Xu, Jiangsu (CN); Liming Tang, Jiangsu (CN); Jianrui Li, Jiangsu (CN); Huiming Cai, Jiangsu (CN)

(73) Assignee: Jinling Hospital, Affiliated Hospital of Medical School, Nanjing University, Jiangsu (CN)

(21) Appl. No.: 18/602,052

(22) Filed: Mar. 12, 2024

(30) Foreign Application Priority Data
Aug. 15, 2023 (CN) 202311020550.5

(51) Int. Cl. G01N 21/65; G01N 21/64; G06T 7/00

(52) U.S. Cl. CPC G01N 21/65; G01N 21/6458; G06T 7/0012; G01N 2201/023; G01N 2201/1293; G01N 2201/1296; G06T 2207/10064; G06T 2207/20081; G06T 2207/20116

(57) ABSTRACT

Disclosed are a molecular imaging method using Raman spectra and system based on machine learning cascade. The system includes a coordinate localization module, a hierarchical clustering analysis module, a Raman predictive imaging module, and a similarity analysis module. An untreated frozen tissue slice is attached to a stainless steel slide, and an adjacent slice is attached to a glass slide. The coordinate localization module can match a Raman white light image of a detection sample attached to the stainless steel slide with an immunohistochemistry (IHC) image of the adjacent slice, such that Raman spectra of a target region are accurately collected. The hierarchical clustering analysis module purifies different types of Raman spectra. A machine learning classifier is configured to build a Raman predictive imaging model. The similarity analysis module can evaluate similarity between a predicted Raman predictive image and the IHC image of the adjacent slice.

### CROSS-REFERENCE TO RELATED APPLICATION

This application claims the priority benefit of China application serial no. 202311020550.5, filed on Aug. 15, 2023. The entirety of the above-mentioned patent application is hereby incorporated by reference herein and made a part of this specification.

### TECHNICAL FIELD

The present disclosure relates to a molecular imaging method and system of Raman spectra, and particularly relates to a molecular imaging method and system of Raman spectra based on machine learning cascade.

### BACKGROUND

At present, molecular imaging is implemented through Raman spectra, which is mainly surface-enhanced Raman scattering (SERS). SERS is a spectroscopic technology based on Raman scattering. An analyte is placed on a surface of a nanostructure having a surface enhancement effect, such that sensitivity of Raman scattering can be significantly improved. This technology has been widely used in analytical chemistry, biomedicine and nanotechnology because it can detect and analyze a trace of molecules and provide a highly sensitive analytical tool. However, it requires to combine heavy metals such as gold, silver and other nano-materials with target molecules, that is, an exogenous probe may be introduced into a body, which will cause unpredictable risks and is complicated in material preparation process.

Stimulated Raman Histology (SRH) of coherent Raman scattering (CRS) can achieve fast imaging in 90 s, but it can only obtain images of a nucleus and cytoplasm, which is similar to hematoxylin-eosin (HE) staining. During the study published in Nat. Med (2023), different molecular types of tissues of glioma were collected through SRH, and then imaging was conducted, which involved wild isocitrate dehydrogenase (IDH) of glioma, IDH mutation, and IDH mutation in combination with 1p/19q co-deletion. However, collection is conducted by patient, so this method cannot distinguish positions where IDH mutation occurs and positions where no mutation occurs. Heterogeneity of gene change caused by subclone mutation is ignored. 93% accuracy can be achieved after training of millions of human glioma images. This method requires a large number of samples and deep learning technology to achieve molecular imaging, and SRH cannot obtain a full Raman peak, so subsequent verification tests cannot be conducted. At present, routine molecular pathological detection takes 2 days, and genetic detection takes about 1 week.

### SUMMARY

Invention objective: an objective of the present disclosure is to provide a rapid and highly accurate molecular imaging method and system of Raman spectra based on machine learning cascade, which do not require too many samples and allow verification tests.

Technical solution: a molecular imaging method of Raman spectra based on machine learning cascade according to the present disclosure includes the following steps:

1, attaching an untreated frozen tissue slice to a stainless steel slide such that a detection sample is obtained, and then attaching an adjacent tissue slice to a glass slide such that a control sample is obtained;

2, independently packaging the detection sample, storing the detection sample at 20° C. or below, conducting immunohistochemistry (IHC) staining on the control sample, obtaining an IHC image, selecting and defining a region of interest (ROI) on the IHC image, placing the stainless steel slide to which the detection sample is attached in a confocal Raman white-light field, obtaining a Raman white light image, and collecting Raman spectra of the ROI in the Raman white light image corresponding to a position of the IHC image;

3, inputting the collected Raman spectra into a hierarchical clustering analysis module, obtaining Raman spectra of different types of biomolecules in the ROI, excluding other types of Raman spectra according to characteristic peaks of different types of Raman spectra, and reserving pure Raman spectra of a target biomolecule in the ROI;

4, respectively inputting different types of obtained Raman spectra in different ROIs into a plurality of machine learning method models for training, obtaining a plurality of machine learning classification models, evaluating the plurality of machine learning classification models, selecting a machine learning classification model having optimal performance for creation of different types of Raman prediction models as a final Raman predictive imaging model, and obtaining a Raman predictive image and a quantitative score of a target biomolecule of the Raman predictive image;

5, evaluating similarity between the IHC image and the Raman predictive image predicted through the Raman predictive imaging model with a similarity analysis module, and evaluating correlation between quantitative scores of target biomolecules of the IHC image and the Raman predictive image, that is, evaluating reliability of the Raman predictive image of the final Raman predictive imaging model; and

6, preprocessing the Raman spectra collected at any position of a sample to be detected, then inputting the preprocessed Raman spectra into the Raman predictive imaging model, and obtaining a Raman image and a quantitative score of a target biomolecule.

Further, selecting and defining the ROI on the IHC image includes the following specific steps:
selecting an anatomical marker point on the IHC image, and coloring the anatomical marker point as a reference point;
defining the ROI around the reference point, where typically, these are areas in the IHC image with strong staining or no staining;
reserving a scale bar and a numerical value of an image of the ROI, reserving the image of the ROI, saving the image as an image file, converting the image file into a binary image, and removing pixels exceeding a threshold;
retrieving a contour in the binary image through a findContours function, and obtaining a vertex position of the ROI with a contour index; and
locating the reference point in the binary image at an origin (0,0), and establishing a two-dimensional coordinate system at the origin.

Computation formulas of vertex coordinates of abounding box of the ROI are as follows:

$${x}_{d} = \frac{{x}_{v} - {x}_{p}}{len ( ruler )} \times scale \\ {y}_{d} = \frac{{y}_{v} - {y}_{p}}{len ( ruler )} \times scale$$

where xᵥ, yᵥ, xₚ, and yₚ denote positions of a vertex v and an origin p of the binary image, respectively, scale denotes the scale bar, len(ruler) denotes a length of the scale bar, and x_{d} and y_{d} denote scaling coordinates of the vertex.

Further, when the Raman white light image is obtained, the stainless steel slide to which the detection sample is attached is placed on a cooling apparatus, and the cooling apparatus is arranged on an objective table of confocal Raman spectra. The cooling apparatus includes a base, a cooling tube arranged on the base, a semiconductor chilling plate arranged on the cooling tube, and a bottom plate configured to bear stainless steel and glass slides. Two ends of the cooling tube are in communication with a pipe of a water cooling device. The cooling apparatus can moisturize and cool the detection sample when the detection sample is collected, such that the protein in the detection sample is prevented from thermal denaturation, the detection sample is prevented from cracking, and an original shape of the detection sample is kept.

Further, collecting the Raman spectra of the ROI corresponding to the position of the IHC image in the Raman white light image includes the following specific steps:
adjusting the IHC image and the Raman white light image, and making the IHC image and the Raman white light image at the same angle; and
keeping the Raman white light image and the IHC image the same in magnification ratio, selecting an origin and ROI vertexes at the same position as the IHC image on the Raman white light image, and collecting the Raman spectra of a corresponding ROI on the Raman white light image.

Further, before the Raman spectra of the ROI are inputted into the hierarchical clustering analysis module, standard Raman spectra of different types of cells or standard proteins are collected, and Raman characteristic peaks of different types of biomolecules are obtained. Before the Raman spectra of the ROI are inputted into the hierarchical clustering analysis module, the Raman spectra of the ROI are preprocessed.

Further, machine learning methods include support vector machine, random forest, linear discriminant analysis, gradient boosting trees, and deep learning.

Further, evaluating the plurality of machine learning classification models includes generating a plurality of types of receiver operating characteristic curves and using an area under the plurality of types of receiver operating characteristic curves as an evaluation index while evaluating performance of the plurality of machine learning classification models with mean sensitivity, specificity and accuracy.

Further, according to staining colors of different target biomarkers in the IHC image, a prediction result of the Raman predictive imaging model is given a corresponding pseudo-color. Frequency of each predicted value of machine learning classification model is computed through a table function (r4.2.2), and then a ratio of the number of different types of Raman spectra to a total Raman spectrum number is obtained through prop according to a table function.

Further, evaluating the reliability of the Raman predictive image of the Raman predictive imaging model includes the following steps:
selecting the ROI from the IHC image, obtaining coordinate values of the ROI, and obtaining the Raman spectra of a corresponding ROI in the Raman white light image according to the coordinate values; and
inputting the collected Raman spectra and Raman predictive image into the similarity analysis module, and evaluating brightness, contrast and structural similarity between the Raman predictive image and the IHC image of the adjacent slice.

$$SSIM = {[ l ( x , y ) ]}^{α} \cdot {[ c ( x , y ) ]}^{β} \cdot {[ s ( x , y ) ]}^{γ} \\ l ( x , y ) = \frac{2 {μ}_{x} {μ}_{x} + {C}_{1}}{{μ}_{x}^{2} + {μ}_{y}^{2} + {C}_{1}} \\ c ( x , x ) = \frac{2 {σ}_{x} {σ}_{y} + {C}_{2}}{{σ}_{x}^{2} + {σ}_{y}^{2} + {C}_{2}} \\ c ( x , y ) = \frac{{σ}_{xy} + {C}_{3}}{{σ}_{x} {σ}_{y} + {C}_{3}}$$

x denotes the IHC image, y denotes the Raman predictive image, l(x,y), c(x,y), and s(x,y) denote brightness comparison, contrast comparison and structure comparison, respectively, μₓ, μ_{y}, σₓ and σ_{y} denote mean intensities and standard deviations of x and y, respectively, C₁, C₂, and C₃ denote constant terms, an exponential condition is set as “α=β=γ=1”, and in consideration that computation of structural similarity (SSIM) is based on a single-color region of the IHC image or the Raman predictive image, a color region is separated through k-means.

On the basis of the same inventive concept, the present disclosure further provides a molecular imaging system of Raman spectra based on machine learning cascade. The system includes:
a coordinate localization module configured to obtain coordinates of ROIs of an IHC image and a Raman white light image;
a hierarchical clustering analysis module configured to conduct classification and purification on the Raman spectra in the ROI and obtain the Raman spectra of a target biomolecule in the ROI;
a Raman predictive imaging module configured to predict a molecular type of a sample to be detected and build a Raman image, and to obtain a Raman predictive image and a quantitative score of a target biomolecule of the Raman predictive image; and
a similarity analysis module configured to evaluate similarity between the Raman predictive image of the Raman predictive imaging module and the IHC image, and to evaluate correlation between quantitative scores of target biomolecules of the Raman predictive image and the IHC image.

Further, according to the coordinate localization module, a stainless steel slide is used as a substrate, an untreated frozen tissue slice is attached to the stainless steel slide and kept at 20° C. or below, then an adjacent tissue slice is attached to a glass slide, the two slices are kept at the same angle, IHC staining is conducted on the tissue slice on the glass slide, the IHC image is obtained, an anatomical marker point is selected on the IHC image and colored as a reference point, a ROI is defined around the reference point, a scale bar and a numerical value of an image of the ROI are reserved, the image of the ROI is reserved, the image is saved as an image file, the image file is converted into a binary image, pixels exceeding a threshold are removed, a contour is retrieved in the binary image through a findContours function, a vertex position of the ROI is obtained with a contour index, the reference point in the binary image is located at an origin (0,0), a two-dimensional coordinate system is established at the origin, and computation formulas of vertex coordinates of a bounding box of the ROI are as follows:

$${x}_{d} = \frac{{x}_{v} - {x}_{p}}{len ( ruler )} \times scale \\ {y}_{d} = \frac{{y}_{v} - {y}_{p}}{len ( ruler )} \times scale$$

where xᵥ, yᵥ, xₚ, and yₚ denote positions of a vertex v and an origin p of the binary image, respectively, scale denotes the scale bar, len(ruler) denotes a length of the scale bar, and x_{d} and y_{d} denote scaling coordinates of the vertex; and the detection sample attached to the stainless steel slide is placed in a confocal Raman white-light field, the Raman white light image is obtained, the Raman white light image and the IHC image are kept to be the same in magnification ratio, an origin and ROI vertexes at the same position as the IHC image are selected on the Raman white light image, and the Raman spectra of a corresponding ROI are collected on the Raman white light image.

According to the hierarchical clustering analysis module, the Raman spectra of other types of biomolecules in the ROI are excluded with the hierarchical clustering analysis module, different types of the Raman spectra are obtained, the other types of the Raman spectra are excluded according to characteristic peaks of the different types of Raman spectra, and pure Raman spectra of a target biomolecule in the ROI are reserved.

According to the Raman predictive imaging module, different types of Raman spectra are firstly predicted with different machine learning method models respectively, a machine learning classification model having optimal performance is selected for creation of Raman prediction models of different types of biomolecules as a final Raman predictive imaging model, then according to staining colors of different target biomolecule markers in the IHC image, a prediction result of the Raman predictive imaging model is given a corresponding pseudo-color, the Raman predictive image is obtained, and proportional scores of the different types of biomolecules are computed according to proportions of different types.

According to the similarity analysis module, the ROI is selected from the IHC image, coordinate values of the ROI are obtained, Raman spectra of a corresponding ROI in the Raman white light image are obtained according to the coordinate values, the collected Raman spectra are preprocessed and then inputted into the Raman predictive imaging model, the Raman predictive image is obtained, the Raman predictive image and an IHC image of an adjacent slice are inputted into the similarity analysis module, and brightness, contrast and structural similarity between the Raman predictive image and the IHC image of the adjacent slice are evaluated.

$$SSIM = {[ l ( x , y ) ]}^{α} \cdot {[ c ( x , y ) ]}^{β} \cdot {[ s ( x , y ) ]}^{γ} \\ l ( x , y ) = \frac{2 {μ}_{x} {μ}_{x} + {C}_{1}}{{μ}_{x}^{2} + {μ}_{y}^{2} + {C}_{1}} \\ c ( x , x ) = \frac{2 {σ}_{x} {σ}_{y} + {C}_{2}}{{σ}_{x}^{2} + {σ}_{y}^{2} + {C}_{2}} \\ c ( x , y ) = \frac{{σ}_{xy} + {C}_{3}}{{σ}_{x} {σ}_{y} + {C}_{3}}$$

x denotes the IHC image. y denotes the Raman predictive image. l(x,y), c(x,y), and s(x,y) denote brightness comparison, contrast comparison and structure comparison, respectively. μₓ, μ_{y}, σₓ and σ_{y} denote mean intensities and standard deviations of x and y, respectively. C₁, C₂, and C₃ denote constant terms. An exponential condition is set as “α=β=γ=1”. In consideration that computation of SSIM is based on a single-color region of the IHC image or the Raman predictive image, a color region is separated through k-means.

Data obtained by preprocessing the Raman spectra collected at any position of the tissue slice of the sample to be detected is inputted into the Raman predictive imaging module, such that the Raman image and a quantitative score of a target molecule are obtained.

Beneficial effects: compared with the prior art, the present disclosure has the following obvious advantages as follows: the Raman spectra of the target biomolecule can be quickly obtained, the obtained Raman spectra are high in accuracy, and meanwhile the quantitative score of the Raman spectra can be obtained; labeling with exogenous probes is not conducted, the Raman spectra has little damage to tissue, and the detected tissue can be used in other experiments; frozen tissue detection is simple in preparation and can be used for clinical fresh in-vitro tissues; and the Raman spectra are rich in molecular information, such that contents of different molecules can be studied, or experimental results can be verified.

### BRIEF DESCRIPTION OF THE DRAWINGS

FIG. 1 is a schematic coordinate diagram of an example of the present disclosure, where 1A of FIG. 1 is a multiplex immunofluorescence (MxIF) image, 1B of FIG. 1 is a Raman white light image, 1C of FIG. 1 is a programmed death ligand 1_{G} (PD-L1_{G}) region, and 1D of FIG. 1 is a PD-L1_{L} region.

FIG. 2 is a schematic diagram of a hierarchical clustering model according to an example of the present disclosure, where 2A of FIG. 2 shows collected Raman spectra of different cells, 2B of FIG. 2 shows mean Raman spectra and characteristic peaks of different cells, 2C of FIG. 2 is collected Raman spectra of a region of interest (ROI) of mouse glioma tissue, 2D of FIG. 2 is a cluster tree diagram of hierarchical clustering analysis, and 2E of FIG. 2 shows mean Raman spectra of different clusters through hierarchical clustering analysis.

FIG. 3 is a schematic diagram of a Raman predictive imaging module of machine learning according to an example of the present disclosure, where 3A of FIG. 3 shows mean classification accuracy of different machine learning classifiers, 3B of FIG. 3 is a confusion matrix diagram of support vector machine (SVM) for 5 different types, 3C of FIG. 3 shows receiver operating characteristic (ROC) curves of SVM for different types, 3D of FIG. 3 is a MxIF image of an adjacent slice in 2C of FIG. 2, 3E of FIG. 3 is an enlarged view of a rectangular box region in 3D of FIGS. 3, and 3F of FIG. 3 is a SVM Raman predictive image.

FIG. 4 is a schematic diagram of a quantitative score according to an example of the present disclosure, where 4A of FIGS. 4 and 4E of FIG. 4 are SVM Raman predictive images, 4B of FIGS. 4 and 4F of FIG. 4 are MxIF, 4C of FIGS. 4 and 4G of FIG. 4 show quantitative scores of PD-L1 expression on the basis of a SVM Raman predictive image and MxIF, and 4D of FIGS. 4 and 4H of FIG. 4 show Pearson correlation analysis of quantitative scores of PD-L1 expression on the basis of a SVM Raman predictive image and MxIF.

FIG. 5 is a schematic diagram of similarity analysis according to an example of the present disclosure, where 5A of FIGS. 5 and 5B of FIG. 5 are MxIF images of a core region of a tumor, 5C of FIG. 5 is a MxIF image of a peripheral region of a tumor, 5D of FIG. 5 is a MxIF image of an infiltrative border between a tumor and normal brain tissue, i of 5A of FIG. 5, i of 5B of FIG. 5, i of 5C of FIG. 5 and i of 5D of FIG. 5 are SVM Raman predictive images, ii of 5A of FIG. 5, ii of 5B of FIG. 5, ii of 5C of FIG. 5 and ii of 5D of FIG. 5 are corresponding MxIF images amplified from boxes in “5A of FIG. 5 to 5D of FIG. 5”, “iii of 5A of FIG. 5 to viii of 5A of FIG. 5”, “iii of 5B of FIG. 5 to viii of 5B of FIG. 5”, “iii of 5C of FIG. 5 to x of 5C of FIG. 5” and “iii of 5D of FIG. 5 to x of 5D of FIG. 5” show different color blocks extracted from colors in “i of 5A of FIG. 5 to ii of 5A of FIG. 5”, “i of 5B of FIG. 5 to ii of 5B of FIG. 5”, “i of 5C of FIG. 5 to ii of 5C of FIG. 5”, and “i of 5D of FIG. 5 to ii of 5D of FIG. 5” through K-means, respectively, and a percentage below the image is a SSIM value of the two images above the percentage.

FIG. 6 is a schematic diagram of application and implementation of a molecular imaging method of Raman spectra based on machine learning cascade according to an example of the present disclosure.

FIG. 7 is a schematic structural diagram of a molecular imaging system according to the present disclosure.

FIG. 8 is a schematic structural diagram of a cooling apparatus according to the present disclosure.

FIG. 9 is a comparison diagram of Raman spectrum signal-noise ratios between a condition of using confocal Raman spectra cooling apparatus and a condition of traditional normal-temperature collection under different air exposure time, where 9A of FIG. 9 is a comparison diagram of Raman signal-noise ratios (SNRs) between normal brain tissue and glioma tissue under different air exposure time; and 9B of FIG. 9 is a comparison diagram between Raman spectrum signal-noise ratios of glioma tissue collected under different Raman integral time.

FIG. 10 shows instance diagrams of ice-cut glioma samples collected with and without a cooling apparatus according to an example of the present disclosure, where 10A of FIG. 10 is an instance diagram of an ice-cut glioma sample collected without a cooling apparatus, and 10B of FIG. 10 is an instance diagram of an ice-cut glioma sample collected with a cooling apparatus.

### DESCRIPTION OF THE EMBODIMENTS

A technical solution of the present disclosure will be further described below with reference to the accompanying drawings.

### Example 1

A molecular imaging method of Raman spectra based on machine learning cascade according to the present disclosure includes the following steps:

(1) An untreated frozen tissue slice is attached to a stainless steel slide such that a detection sample is obtained, and then an adjacent tissue slice is attached to a glass slide such that a control sample is obtained. The stainless steel slide is preferably made of 304 mirror stainless steel, and preferably has a size of 7.5 cm*2.5 cm*2 mm. The stainless steel slide is weak in Raman signals of a substrate and high in signal-noise ratio of Raman signals of the tissue slice. The tissue slice has a thickness of 3 μm-10 μm.

(2) The detection sample and the control sample are separately treated as follows:

(21) The detection sample is independently packaged and stored at 20° C. or below.

(22) Immunohistochemistry (IHC) staining is conducted on the control sample (IHC staining refers to immunohistochemical staining, which is a technology for detecting and locating specific antigens or protein in tissue by labeling target protein with specific antibodies and visualizing the protein with fluorescent stain or enzyme markers), an IHC image is obtained, an anatomical marker point is selected on the IHC image and colored as a reference point, and then a region of interest (ROI) is defined around the reference point. A common shape of the ROI is a rectangle, a circle, a triangle, etc. In order to adapt to an actual detection situation, a shape of the ROI may also be irregular, and the number of ROIs may be one or more. A scale bar and a numerical value of an image of the ROI are reserved, the image of the ROI is reserved, the image is saved as an image file, the image file is converted into a binary image, and pixels exceeding a threshold are removed. A contour is retrieved in the binary image through a findContours function, a vertex position of the ROI is obtained with a contour index, the reference point in the binary image is located at an origin (0,0), and a two-dimensional coordinate system is established at the origin. Computation formulas of vertex coordinates of a bounding box of the ROI are as follows:

$${x}_{d} = \frac{{x}_{v} - {x}_{p}}{len ( ruler )} \times scale \\ {y}_{d} = \frac{{y}_{v} - {y}_{p}}{len ( ruler )} \times scale$$

where xᵥ, yᵥ, xₚ, and yₚ denote positions of a vertex v and an origin p of the binary image, respectively, scale denotes the scale bar, len(ruler) denotes a length of the scale bar, and x_{d} and y_{d} denote scaling coordinates of the vertex.

(23) The stainless steel slide to which the detection sample is attached is placed in a confocal Raman white-light field, and a Raman white light image is obtained. During slicing, the slice attached to the stainless steel slide and the slice attached to the glass slide are kept in a consistent direction. If the two slices are in inconsistent directions, the IHC image or the Raman white light image may be adjusted to be at the same angle. The images may be completely overlapped, and then the Raman white light image and the IHC image are kept to be the same in magnification ratio. An origin and ROI vertexes at the same position as the IHC image are selected on the Raman white light image, and Raman spectra of a corresponding ROI are collected on the Raman white light image.

(3) Standard Raman spectra of different types of cells or standard proteins are collected, and Raman characteristic peaks of different types of biomolecules are obtained. The Raman spectra of the ROI are subjected to preprocessing, where the preprocessing includes cosmic ray removal, baseline calibration, data normalization, etc. The preprocessed Raman spectra of the ROI of the Raman white light image are inputted into a hierarchical clustering analysis module, such that different types of Raman spectra in the ROI are obtained. Other types of Raman spectra are excluded according to characteristic peaks of different types of Raman spectra, and pure Raman spectra of a target biomolecule in the ROI are reserved.

(4) Different types of obtained Raman spectra in different ROIs are respectively inputted into a plurality of machine learning method models for training, and a plurality of machine learning classification models are obtained. The machine learning methods include, but are not limited to, support vector machine, random forest, linear discriminant analysis, gradient boosting trees, and deep learning. Then, the plurality of machine learning classification models are evaluated. A plurality of types of receiver operating characteristic curves are generated and an area under the plurality of types of receiver operating characteristic curves is used as an evaluation index while performance of the machine learning classification model is evaluated with mean sensitivity, specificity and accuracy. A machine learning classification model having optimal performance is selected for creation of different types of Raman prediction models as a final Raman predictive imaging model, and a Raman predictive image is obtained. In order to visually view a final Raman predictive imaging condition, according to staining colors of different target biomarkers in the IHC image, a prediction result of the Raman predictive imaging model may be given a corresponding pseudo-color. In order to further visually view the final Raman predictive imaging condition, quantitative scores of target biomolecules of the Raman predictive image are obtained, and proportional scores of the different types of biomolecules are computed according to proportions of different types. Firstly, frequency of each predicted value of machine learning classification model is computed through a table function (r4.2.2), and then a ratio of the number of different types of Raman spectra to a total Raman spectrum number is obtained through prop according to a table function.

(5) Similarity between the IHC image and the Raman predictive image of the Raman predictive imaging module is evaluated with a structural similarity (SSIM) module, and correlation between quantitative scores of target biomolecules of the Raman predictive image and the IHC image is evaluated. That is, reliability of the Raman predictive image of the Raman predictive imaging model is evaluated. Firstly, the ROI is selected from the IHC image, coordinate values of the ROI are obtained, Raman spectra of a corresponding ROI in the Raman white light image are obtained according to the coordinate values. The collected Raman spectra and the Raman predictive image are inputted into the similarity analysis module, and brightness, contrast and structural similarity between the Raman predictive image and the IHC image of the adjacent slice are evaluated.

$$SSIM = {[ l ( x , y ) ]}^{α} \cdot {[ c ( x , y ) ]}^{β} \cdot {[ s ( x , y ) ]}^{γ} \\ l ( x , y ) = \frac{2 {μ}_{x} {μ}_{x} + {C}_{1}}{{μ}_{x}^{2} + {μ}_{y}^{2} + {C}_{1}} \\ c ( x , x ) = \frac{2 {σ}_{x} {σ}_{y} + {C}_{2}}{{σ}_{x}^{2} + {σ}_{y}^{2} + {C}_{2}} \\ c ( x , y ) = \frac{{σ}_{xy} + {C}_{3}}{{σ}_{x} {σ}_{y} + {C}_{3}}$$

x denotes the IHC image. y denotes the Raman predictive image. l(x,y), c(x,y), and s(x,y) denote brightness comparison, contrast comparison and structure comparison, respectively. μₓ, μ_{y}, σₓ, and σ_{y} denote mean intensities and standard deviations of x and y, respectively. C₁, C₂, and C₃ denote constant terms. An exponential condition is set as “α=β=γ=1”. In consideration that computation of SSIM is based on a single-color region of the IHC image or the Raman predictive image, a color region is separated through k-means (OpenCV, python 3.6.5). Correlation between biomolecular proportional scores of the Raman predictive image and the IHC image is analyzed through Pearson correlation analysis.

(6) Raman spectra collected at any position of a sample to be detected are preprocessed and then inputted into the Raman predictive image, and a Raman image and a quantitative score of a target biomolecule are obtained.

### Example 2

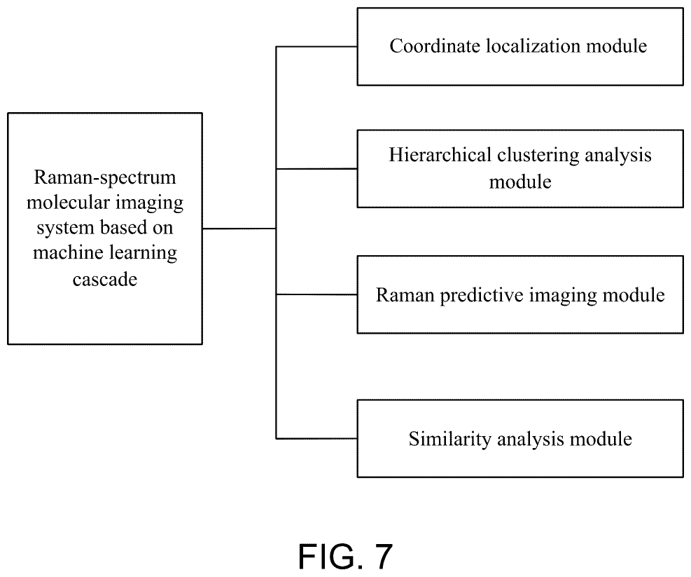

As shown in FIG. 7, a molecular imaging system of Raman spectra based on machine learning cascade according to the present disclosure includes:
a coordinate localization module configured to obtain coordinates of ROIs of an IHC image and a Raman white light image. Specifically, a stainless steel slide is used as a substrate, an untreated frozen tissue slice is attached to the stainless steel slide and kept at 20° C. or below. Then an adjacent tissue slice is attached to a glass slide. The two slices are kept at the same angle. IHC staining is conducted on the tissue slice on the glass slide, and the IHC image is obtained. An anatomical marker point is selected on the IHC image and colored as a reference point. A ROI is defined around the reference point. A scale bar and a numerical value of an image of the ROI are reserved, the image of the ROI is reserved, and the image is saved as an image file. The image file is converted into a binary image. Pixels exceeding a threshold are removed. A contour is retrieved in the binary image through a findContours function. A vertex position of the ROI is obtained with a contour index. The reference point in the binary image is located at an origin (0,0), and a two-dimensional coordinate system is established at the origin. Computation formulas of vertex coordinates of a bounding box of the ROI are as follows:

$${x}_{d} = \frac{{x}_{v} - {x}_{p}}{len ( ruler )} \times scale \\ {y}_{d} = \frac{{y}_{v} - {y}_{p}}{len ( ruler )} \times scale$$

where xᵥ, yᵥ, x_{y}, and yₚ denote positions of a vertex v and an origin p of the binary image, respectively, scale denotes the scale bar, len(ruler) denotes a length of the scale bar, and x_{d} and y_{d} denote scaling coordinates of the vertex. The detection sample attached to the stainless steel slide is placed in a confocal Raman white-light field, and the Raman white light image is obtained. the Raman white light image and the IHC image are kept to be the same in magnification ratio. An origin and ROI vertexes at the same position as the IHC image are selected on the Raman white light image. Raman spectra of a corresponding ROI are collected on the Raman white light image.

The system further includes a hierarchical clustering analysis module configured to conduct classification and purification on Raman spectra in the ROI and obtain Raman spectra of a target biomolecule in the ROT. Specifically, Raman spectra of other types of biomolecules in the ROI are excluded with the hierarchical clustering analysis module, different types of Raman spectra are obtained, the other types of Raman spectra are excluded according to characteristic peaks of the different types of Raman spectra, and pure Raman spectra of a target biomolecule in the ROI are reserved.

The system further includes a Raman predictive imaging module configured to predict a molecular type of a sample to be detected and build a Raman image. Specifically, different types of Raman spectra are firstly predicted with different machine learning method models respectively, a machine learning classification model having optimal performance is selected for creation of Raman prediction models of different types of biomolecules as a final Raman predictive imaging model. Then, according to staining colors of different target biomolecule markers in the IHC image, a prediction result of the Raman predictive imaging model is given a corresponding pseudo-color, and the Raman predictive image is obtained. Proportional scores of the different types of biomolecules are computed according to proportions of different types. A quantitative score of a target biomolecule of the Raman predictive image is obtained.

The system further includes a similarity analysis module configured to evaluate similarity between the Raman predictive image predicted through the Raman predictive imaging module and the IHC image and give the quantitative score of the Raman predictive image. Specifically, the ROI is selected from the IHC image, and coordinate values of the ROI are obtained. Raman spectra of a corresponding ROI in the Raman white light image are obtained according to the coordinate values. The collected Raman spectra are preprocessed and then inputted into the Raman predictive imaging model, and the Raman predictive image is obtained. The Raman predictive image and an IHC image of an adjacent slice are inputted into the similarity analysis module. Brightness, contrast and structural similarity between the Raman predictive image and the IHC image of the adjacent slice are evaluated.

$$SSIM = {[ l ( x , y ) ]}^{α} \cdot {[ c ( x , y ) ]}^{β} \cdot {[ s ( x , y ) ]}^{γ} \\ l ( x , y ) = \frac{2 {μ}_{x} {μ}_{x} + {C}_{1}}{{μ}_{x}^{2} + {μ}_{y}^{2} + {C}_{1}} \\ c ( x , x ) = \frac{2 {σ}_{x} {σ}_{y} + {C}_{2}}{{σ}_{x}^{2} + {σ}_{y}^{2} + {C}_{2}} \\ c ( x , y ) = \frac{{σ}_{xy} + {C}_{3}}{{σ}_{x} {σ}_{y} + {C}_{3}}$$

x denotes the IHC image. y denotes the Raman predictive image. i(x,y), c(x,y), and s(x,y) denote brightness comparison, contrast comparison and structure comparison, respectively. μₓ, μ_{y}, σₓ and σ_{y} denote mean intensities and standard deviations of x and y, respectively. C₁, C₂, and C₃ denote constant terms. An exponential condition is set as “α=β=γ=1”. In consideration that computation of SSIM is based on a single-color region of the IHC image or the Raman predictive image, a color region is separated through k-means.

During actual application, data obtained by preprocessing Raman spectra collected at any position of a tissue slice of the sample to be detected only needs to be inputted into the Raman predictive imaging module, such that a Raman image and a quantitative score of a target molecule may be obtained, which does not require the coordinate localization module, the hierarchical clustering analysis module and the similarity analysis module.

### Example 3

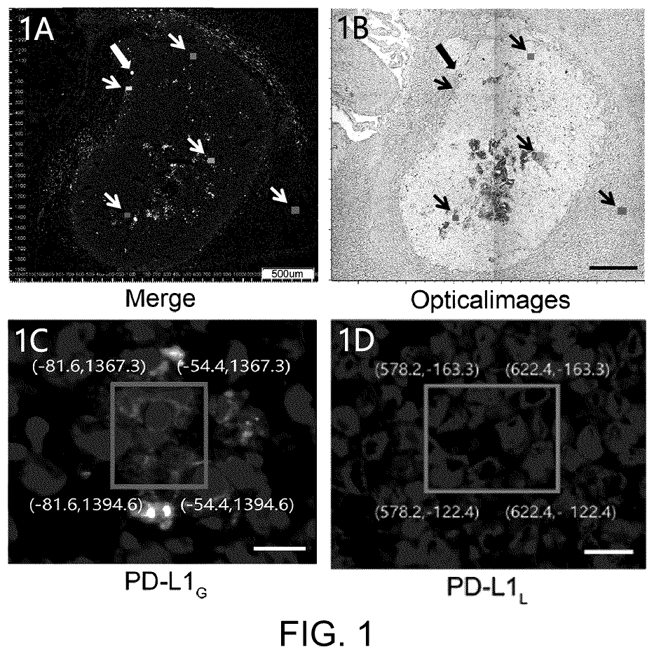

As shown in FIGS. 1-6, the present disclosure was applied to a case of predicting Raman spectrum images of programmed death ligand-1 (PD-L1) of a tumor cell and an immune cell as follows:

Glioblastoma (GBM) was a highly infiltrative and location-specific brain tumor, which was limited in treatment options and poor in prognosis. Surgical treatment was a main treatment means for a GBM patient. Postoperative immunotherapy was expected to improve a survival rate of GBM patients. An expression level of programmed death ligand-1 in the tumor cell and the immune cell in an immune microenvironment (IME) was a main predictive index of efficacy of immunotherapy. However, expression of the PD-L1 in the IME had significant heterogeneity, and was inconsistent even in the same tissue, which brought challenges to response prediction of postoperative immunotherapy. Therefore, visualization of an expression level of PD-L1 in a residual GBM IME in a key brain functional region during operation was important for making an optimal treatment strategy between tumor resection and immunotherapy. However, at present, immunohistochemistry (IHC) staining was a main method for molecular detection in histopathology, which was configured to detect and locate specific antigens or protein in tissue by labeling target protein with specific antibodies and visualizing the protein with fluorescent stain or enzyme markers. An incubation process of antigens and antibodies was involved, which had many steps and consumes a long time. The process generally took 2 days for completion. A Raman spectrum image of PD-L1 was predicted through the method of the present disclosure, such that heterogeneity of GBM intratumoral modulation therapy (IMT) can be overcome. Expression levels of PD-L1 in a glioma cell, CD8+T cells, a macrophage and a normal cell in the GBM IMT can be visualized, and a tumor/normal brain infiltrative border can be accurately defined.

Firstly, an in-situ glioma model of 8 C57BL/6 mice implanted with GL261 cells was created. About 25 days later, magnetic resonance imaging (MRI) of mice proved that glioma was successfully implanted in situ, and brains of mice were taken out after hearts of the mice were perfusion with normal saline. An optimal cutting temperature (OCT) agent was embedded in brain tissue, and then the tissue was quickly frozen with liquid nitrogen and sliced with a cryotome into slices having a thickness of 5 μm. A slice was attached to a customized stainless steel slide, such that a detection sample was obtained. An adjacent slice was attached to a glass slide, such that a control sample was obtained. Multiplex immunofluorescence (MxIF) staining was conducted on one control sample. The MxIF staining was a type of IHC staining. The tissue slice on the stainless steel slide was independently packaged and stored in a refrigerator at −80° C., such that the problem that internal and external exchange of substances changes properties of substances in the tissue is prevented.

An anatomical marker point was selected on a MxIF image, and colored as a colored dot, as shown by a white arrow in 1A of FIG. 1A (a scale bar of 1A and 1B of FIG. 1 was 500 μm). Different ROIs were selected, which included a high expression region PD-L1_{G} of PD-L1 in glioma, a high expression region PD-L1_{T} of PD-L1 in CD8+T cells, a high expression region PD-L1_{M} of PD-L1 in a macrophage, a low expression region PD-L1L of PD-L1 in glioma, and a normal brain tissue region. The ROI was defined with a rectangle. A scale bar and a numerical value were reserved, a current interface was saved as an image file, the image file was converted into a binary image, and pixels exceeding a threshold were removed. A contour was retrieved in the binary image through a findContours function, and a vertex position of the rectangle of the ROI was obtained with a contour index. A colored dot was retrieved in the binary image and determined as an origin (0,0), and a two-dimensional coordinate system was established according to the origin. A length and a numerical value of the scale bar at a lower right corner of the binary image were retrieved. Vertex coordinates of a bounding box were as shown in 1C and 1D of FIG. 1 (a scale bar of 1C and 1D of FIG. 1 was 10 μm), and may be computed according to the distance and scale bar. A formula was as follows:

$${x}_{d} = \frac{{x}_{v} - {x}_{p}}{len ( ruler )} \times scale \\ {y}_{d} = \frac{{y}_{v} - {y}_{p}}{len ( ruler )} \times scale$$

xᵥ, yᵥ, xₚ, and yₚ denoted positions of a vertex v and an origin p of a rectangular box in a pixel image, respectively, scale denoted the scale bar, len(ruler) denoted a length of the scale bar at the lower right corner, and x_{d} and y_{d} denoted scaling coordinates of the vertex. In some cases, when angles of the MxIF image and a Raman microscope white-light image were inconsistent, the angle of the MxIF image was adjusted to make the two angles consistent.

When Raman spectra were collected, the detection sample attached to the stainless steel slide was placed in a confocal microscope Raman white-light field, and an anatomical marker point corresponding to the MxIF image was selected, which was as shown by a black arrow in 1B of FIG. 1 and labeled as the origin (0,0). According to the coordinate values of the rectangular box of the ROI, Raman spectra were collected in a ROI corresponding to a Raman white light image. In the experiment, 5029 Raman spectra were collected from different ROIs, and were labeled as 5 sub-groups of PD-L1_{G}, PD-L1_{T}, PD-L1_{M}, PD-L1_{L}, and normal brain tissue.

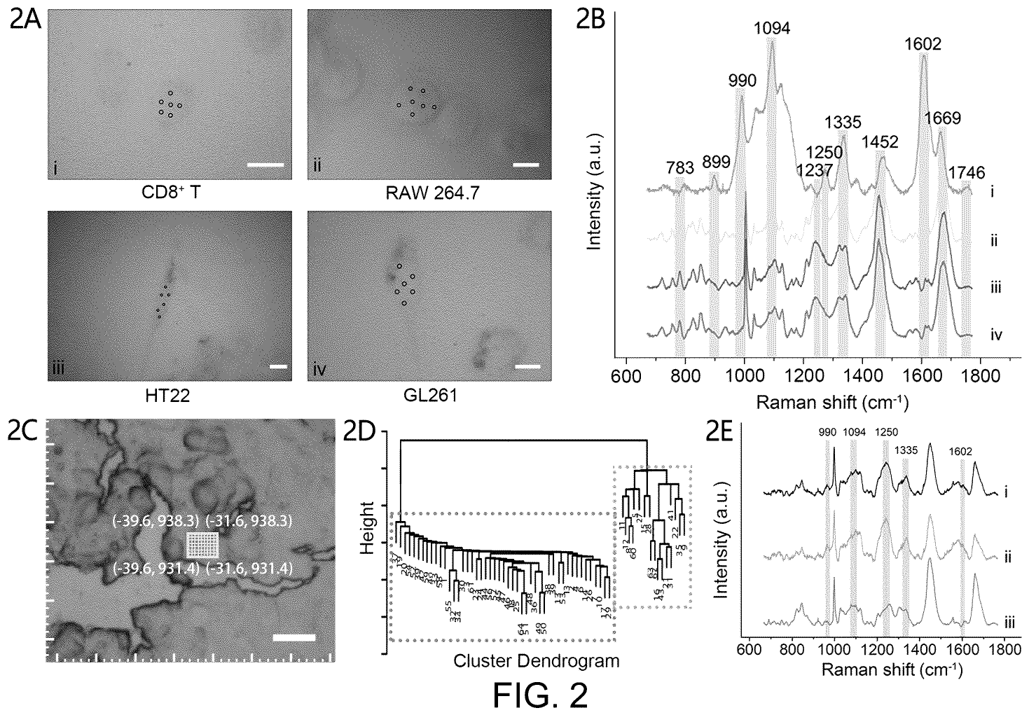

In order to exclude other types of Raman spectra more accurately, standard Raman spectra were collected from mouse CD8+T cells, mouse macrophages RAW264.7, mouse neuron HT22 cells and mouse GL261 glioma cells as reference Raman spectra. Adherent cells (RAW264.7, HT22 and GL261) were cultured in a DMEM medium for 3 generations and then adhered to the stainless steel slide for incubation for 24 hours. After suspension cells (CD8+T) were cultured in a RPMI-1640 medium for 48 hours, a phosphate-buffered saline (PBS) suspension (with a density of 5×10⁵) containing CD8+T cells was prepared and applied to the stainless steel slide. Surfaces of the above 4 types of cells were each covered with a thin layer of PBS, such that Raman spectra of the cells were collected in vivo. 6-8 points were randomly collected on each cell, and averagely 40 spectra were collected for each type of cells, as shown in 2A of FIG. 2 (a scale bar of FIG. 2 was 10 μm). Mean spectra were as shown in 2B of FIG. 2.

The Raman spectra of the ROI were collected as shown in 2C of FIG. 2. Then, the collected Raman spectra data were preprocessed as follows:
(a) quality control was conducted as follows: spectra were collected strictly according to an expression region of PD-L1, and spectra having a signal-noise ratio smaller than 3 were excluded;
(b) cosmic ray removal was conducted as follows: a nearest neighbor algorithm (with a noise level: 0.16, and a spectral height: 5.33) was used to remove a peak that may come from cosmic rays or charge-coupled device (CCD) overload;
(c) baseline correction was conducted as follows: intelligent polynomial fitting (with a polynomial order of 11, and a noise tolerance of 1.5) was used to remove a background Raman signal from a fluorescent background of a stainless steel substrate or sample; and
(d) normalization was conducted as follows: a Raman peak of phenylalanine at 1003 cm⁻¹ was used to normalize the whole Raman spectra.

The preprocessed Raman spectra were inputted into a hierarchical clustering analysis module specifically as follows:
(a) the preprocessed Raman spectrum data was imported into R (version 4.2.2);
(b) a Euclid method of a distance function was used to compute a distance matrix between all Raman spectrum data and reflect the distance matrix on a y axis of a cluster tree diagram;
(c) the distance matrix was used as input, and hierarchical clustering analysis (HCA) was conducted through a single clustering method in a hclust function;
(d) a Plot function was used to draw a cluster tree, and Raman spectrum data of other regions were screened according to the distances between branches and a main cluster, as shown in 2D of FIG. 2; and
(e) according to characteristic peaks of standard Raman spectra of different cells (as shown in 2B of FIG. 2) and mean Raman spectra of different clusters in hierarchical clustering analysis, other types of Raman spectra (as shown in 2E of FIG. 2) were excluded, and pure Raman spectra of a target biomarker in the ROI were reserved; and through the hierarchical clustering analysis module, 352 Raman spectra were removed, with 1294 Raman spectra in a PD-L1_{G} group, 721 Raman spectra in a PD-L1_{T} group, 638 Raman spectra in a PD-L1_{M} group, 1058 Raman spectra in a PD-L1_{L} group, and 966 Raman spectra in normal brain tissue reserved.

Through the above steps, different types of pure Raman spectra were obtained. The used different machine learning methods included support vector machine (SVM), random forest (RF), linear discriminant analysis (LDA), and gradient boosting trees (GBT). The machine learning methods may extract useful signals from complex Raman spectra and use the signals to classify different types of Raman spectra. Classification performance of a machine learning classification model was tested on a data set with mean sensitivity, specificity and accuracy. In addition, a plurality of types of receiver operating characteristic (ROC) curves were used as measure indexes of accuracy of the machine learning classification model, such that a machine learning classification model having optimal classification efficiency was screened for subsequent Raman imaging.

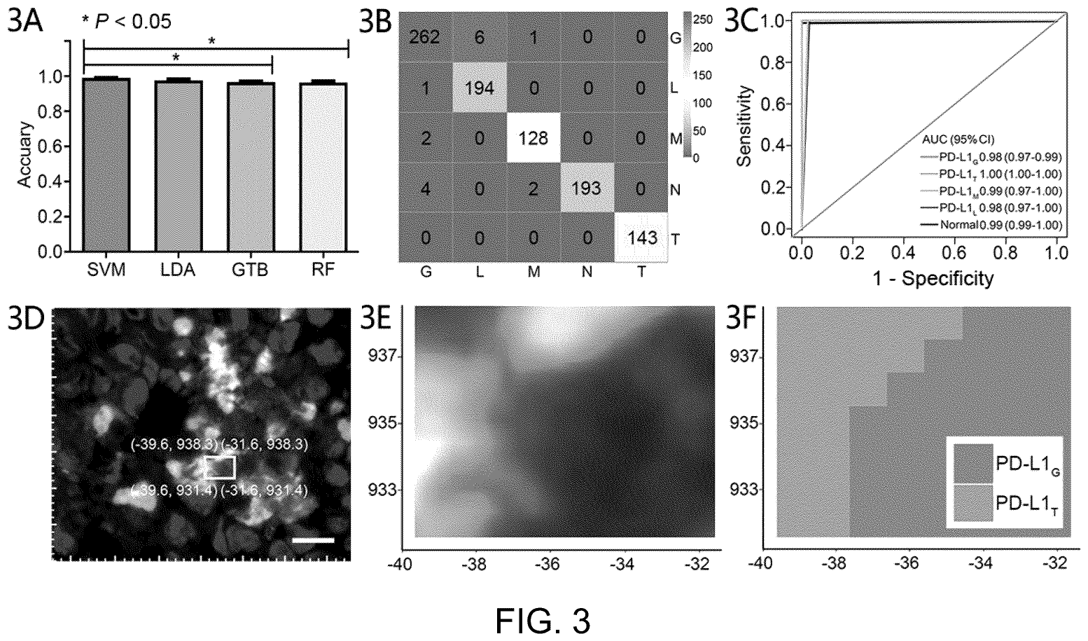

The experimental results showed that a support vector machine (SVM) algorithm had an optimal classification effect on 5 types of PD-L1 expression cells in glioma tissue, and can achieve mean accuracy of 0.990 (as shown in 3A of FIG. 3). The support vector machine (SVM) algorithm was used to build different types of Raman predictive imaging models as a final Raman predictive imaging model. A confusion matrix diagram showed identification errors of 3 spectra in PD-L1_{M}, 7 spectra in PD-L1_{G} and 6 spectra in PD-L1_{L} (as shown in 3B of FIG. 3). The ROC curve showed that classification accuracy of PD-L1_{T} is the highest (1.000), followed by normal brain tissue (0.996), PD-L1_{M} (0.987), PD-L1_{L} (0.984), and PD-L1_{G} (0.982) (as shown in 3C of FIG. 3).

According to colors expressed by different PD-L1 in an adjacent MxIF image (as shown in 3D and 3E of FIG. 3) of the slice, a prediction result of the support vector machine (SVM) was given a corresponding pseudo-color, and a Raman predictive image of the support vector machine (SVM) was built (as shown in 3F of FIG. 3). As shown in FIG. 3, a SVM Raman predictive image was highly consistent with a corresponding MxIF image.

In addition, according to proportions of different types, a tumor proportion score (TPS), a tumor proportion score (TPS) and a cellular composition score (CCS) were computed to quantitatively evaluate expression levels of PD-L1 in glioma cells and surrounding immune cells in GBM IMT. Firstly, frequency of a predicted value of the support vector machine (SVM) was computed through a table function (r4.2.2), and then prop was conducted. A ratio of the number of different types of cells to a total cell number was obtained through a Table function, which was CCS_{Raman}. Computation formulas of TPS_{Raman} and CPS_{Raman} were as follows:

$${TPS}_{Raman} = \frac{PD - L {1}_{G}}{( PD - L {1}_{G} + PD - L {1}_{L} )} \\ {CPS}_{Raman} = \frac{PD - L {1}_{G} + PD - L {1}_{T} + PD - L {1}_{M}}{( PD - L {1}_{G} + PD - L {1}_{L} )} \times 100$$

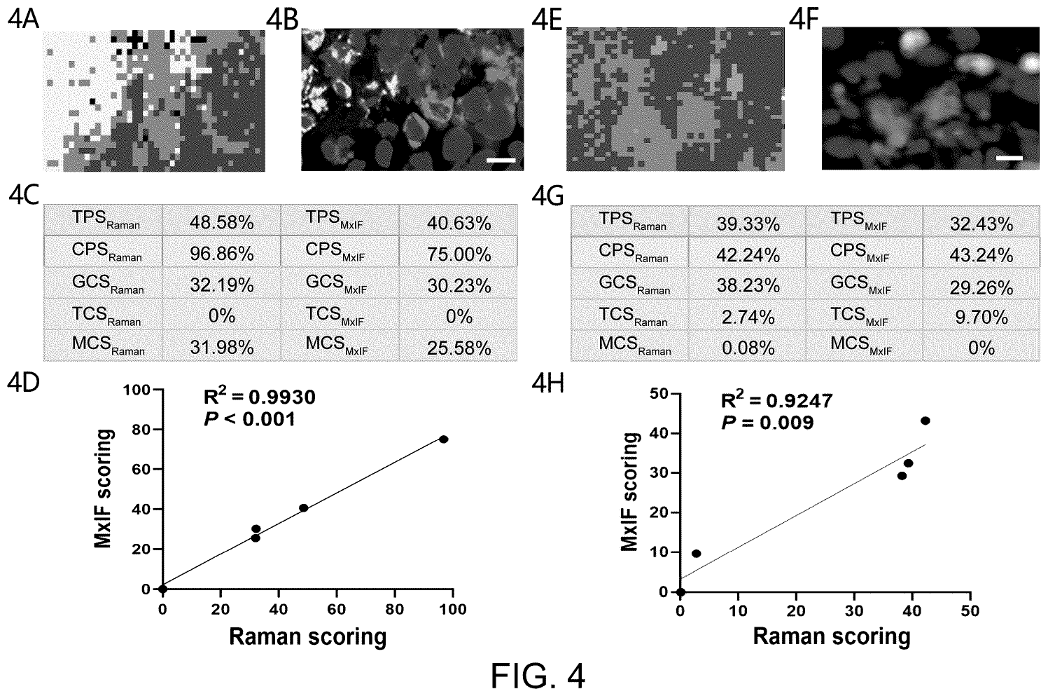

A traditional score based on MxIF was evaluated by two pathologists, and a mean value of two evaluated scores was used. Representative SVM Raman predictive images 1 and 2 and corresponding MxIF images were as shown in 4A, 4B, 4E and 4F of FIG. 4. Quantitative scores of PD-L1 expression of the SVM Raman predictive image and MxIF were as shown in 4C and 4D of FIG. 4. Pearson correlation analysis showed that the Raman predictive image was highly correlated with the MxIF images (R2>0.92, P<0.001), which indicated that the quantitative score of PD-L1 expression in the SVM Raman predictive images had a high correlation with a traditional pathologist score, as shown in FIG. 4.

In the MxIF images, C showed quantitative scores of PD-L1 expression of a SVM Raman predictive image and MxIF, D showed correlation analysis of the quantitative scores of the PD-L1 expression of the SVM Raman predictive image and MxIF, E was a representative SVM Raman predictive image 2, F was a corresponding MxIF image, G showed quantitative scores of PD-L1 expression of a SVM Raman predictive image and MxIF, and H showed correlation analysis of the quantitative scores of the PD-L1 expression of the SVM Raman predictive image and MxIF, with a scale bar of 10 μm.

4856 Raman spectral imaging data were collected from a 2 two C57BL/6 mice in-situ glioma model built in other batches as external verification data, and similarity analysis was conducted to evaluate similarity between a SVM Raman predictive image and an adjacent MxIF image, such that authenticity and robustness of the model were verified.

Specifically, the ROI was firstly selected from the MxIF image, coordinate values of the ROI were obtained, and Raman spectrum imaging data of a corresponding position was collected under confocal Raman microscope white light according to the coordinate values. The collected Raman spectra and Raman predictive image were inputted into the similarity analysis module, and brightness, contrast and structural similarity between the SVM Raman predictive image and the MxIF image of the adjacent slice were evaluated through SSIM, which were defined as follows:

$$SSIM = {[ l ( x , y ) ]}^{α} \cdot {[ c ( x , y ) ]}^{β} \cdot {[ s ( x , y ) ]}^{γ} \\ l ( x , y ) = \frac{2 {μ}_{x} {μ}_{y} + {C}_{1}}{{μ}_{x}^{2} + {μ}_{y}^{2} + {C}_{1}} \\ c ( x , y ) = \frac{2 {σ}_{x} {σ}_{y} + {C}_{2}}{{σ}_{x}^{2} + {σ}_{y}^{2} + {C}_{2}} \\ s ( x , y ) = \frac{{σ}_{xy} + {C}_{3}}{{σ}_{x} {σ}_{y} + {C}_{3}}$$

x denoted the MxIF image. y denoted a confocal Raman microscope white-light image. l(x,y), c(x,y), and s(x,y) denoted brightness comparison, contrast comparison and structure comparison, respectively. μₓ, μ_{y}, σₓ, and σ_{y} denoted mean intensities and standard deviations of x and y, respectively. In the study, in order to prevent a denominator from being 0, constant terms C₁, C₂ and C₃ were set to avoid formula imbalance. In addition, exponents were generally set to satisfy “α=β=γ=1”. In consideration that computation of SSIM was based on a single-color region of MxIF or the SVM Raman predictive image, a color region was separated through k-means (OpenCV, python 3.6.5.).

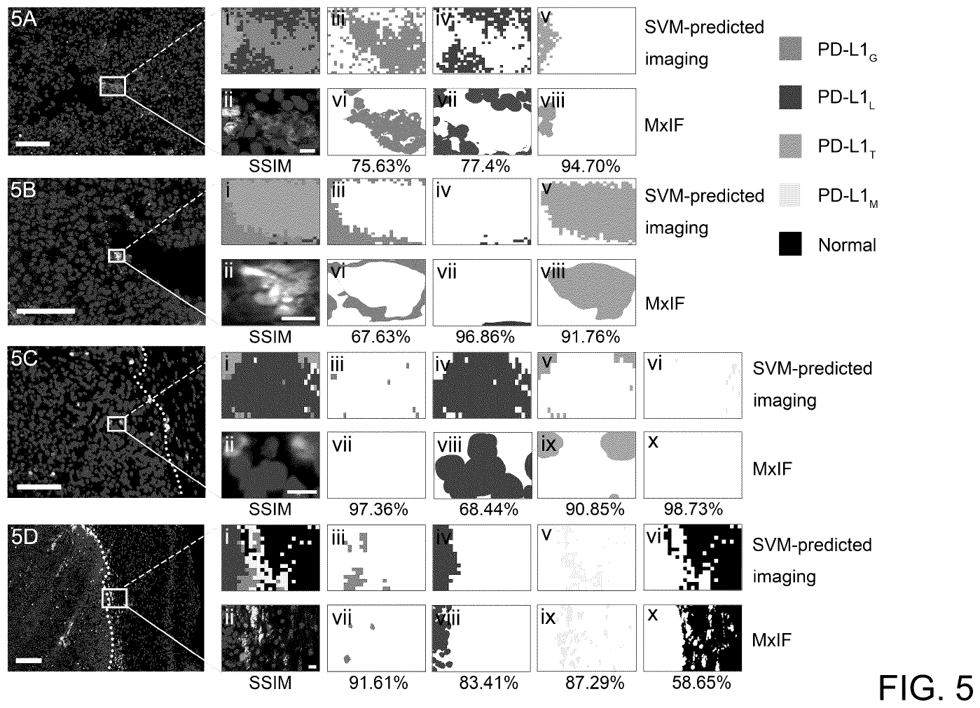

In a core region of glioma, a SVM Raman predictive image clearly distinguished PD-L1_{G} and PD-L1_{T} distributed in an aggregated manner. An imaging result was highly similar to a corresponding MxIF image (mean SSIM was 84.00%, and as shown in 5A of FIG. 5 to viii of 5B of FIG. 5, scale bars of 5A of FIG. 5, 5B of FIG. 5, 5C of FIGS. 5 and 5D of FIG. 5 were 100 μm, and scale bars of ii of 5A of FIG. 5, ii of 5B of FIG. 5, ii of 5C of FIG. 5, and ii of 5D of FIG. 5 were 10 μm). In a peritumoral region, a SVM Raman predictive image showed that PD-L1_{T} was distributed in a scattered manner, which was significantly consistent with MxIF (mean SSIM was 88.85%, and as shown in 5C of FIG. 5 to x of 5C of FIG. 5, dotted lines in 5C of FIG. 5 to 5D of FIG. 5 represented a border). More PD-L1_{M} (mean SSIM was 80.24%, as shown in 5D of FIG. 5 to x of 5D of FIG. 5) existed at an infiltrative border between tumor and normal tissue. Mean SSIM of each type of SVM predictive images was 84.31%, and SSIM of PD-L1_{T} was the highest, which was 92.44%. Generally, the SVM Raman predictive image can describe a molecular border of PD-L1 between different cell types and an infiltrative border between tumor and normal brain tissue. An imaging result was highly similar to a corresponding MxIF image, which indicated significant heterogeneity in spatial distribution of PD-L1 expression cells.

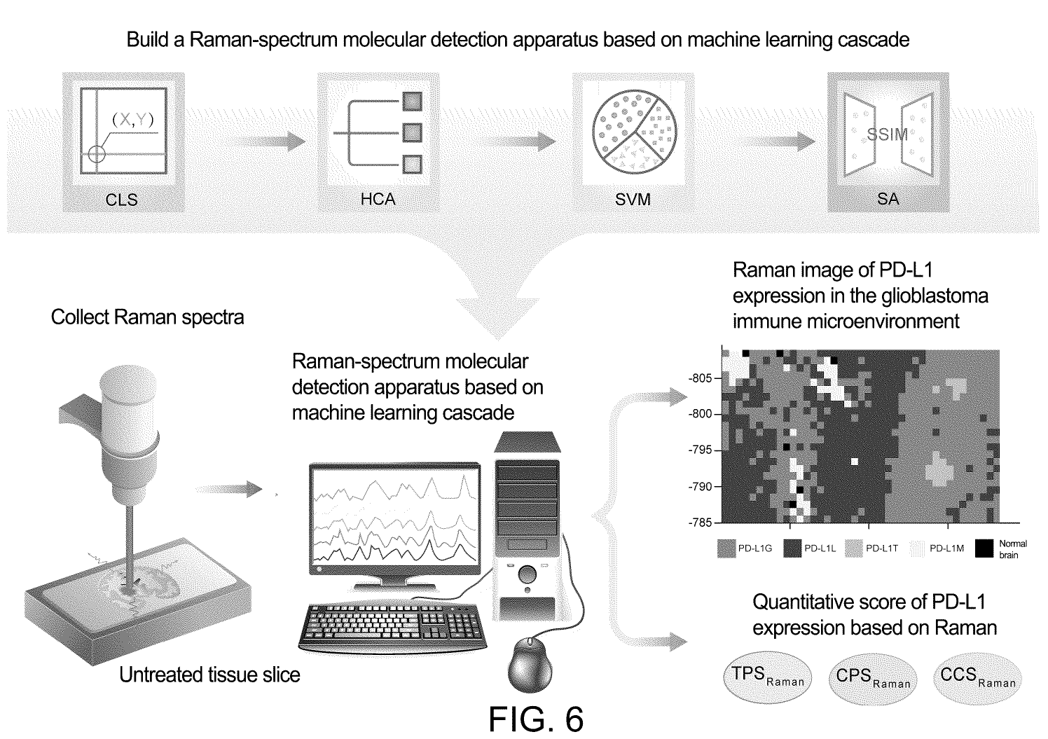

Finally, a Raman image of PD-L1 in GBM IME and quantitative scores of the PD-L1, including TPS_{Raman}, CPS_{Raman} and CCS_{Raman}, can be obtained by directly inputting preprocessed data of Raman spectra of any region of an untreated frozen slice of glioma into the final Raman predictive imaging model (as shown in FIG. 6).

### Example 4

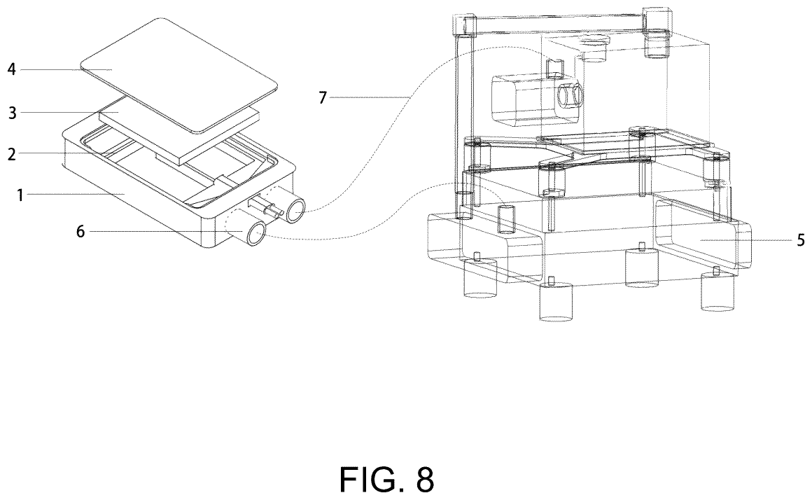

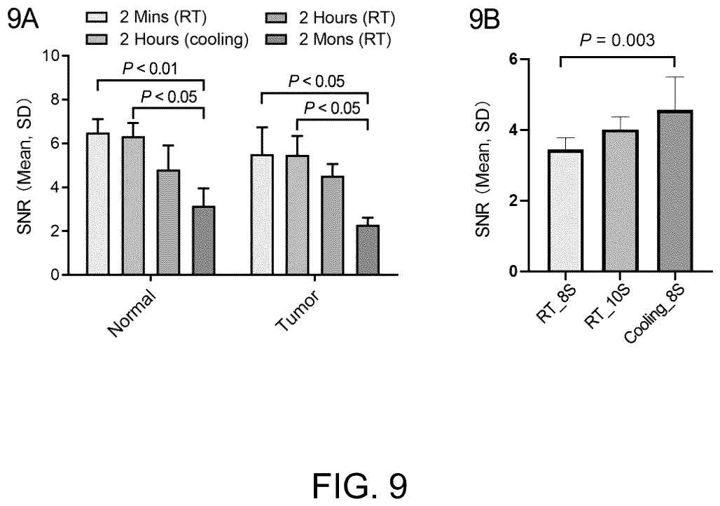

As shown in FIGS. 8-10, when existing confocal Raman spectra is collected, a detection sample is exposed to air, and a surface of the detection sample is dehydrated and cracked due to surface laser irradiation, such that a signal-noise ratio of Raman spectra is reduced, and meanwhile, a shape of the sample is influenced. In addition, a thermal effect of laser may lead to protein degeneration of the detection sample, which makes detection accuracy of the Raman spectra decline. Therefore, in the example, a cooling apparatus is used to cool the detection sample during confocal Raman spectra collection.

When a Raman white light image is obtained, a stainless steel slide to which the detection sample is attached is placed on the cooling apparatus, and the cooling apparatus is arranged on an objective table of confocal Raman spectra. The cooling apparatus includes a base 1, a cooling tube 2 arranged on the base 1, a semiconductor chilling plate 3 arranged on the cooling tube 2, and a bottom plate 4 configured to bear stainless steel and glass slides. The bottom plate 4 is preferably an aluminum plate. Two ends of the cooling tube 2 are in communication with a pipe of a water cooling device 5. The base 1 is provided with two connecting tubes 6. The two ends of the cooling tube 2 are connected to the two connecting tubes 6 respectively. The other ends of the two connecting tubes 6 are both connected to water guide hoses 7. The other ends of the two water guide hoses 7 are connected to a water inlet and a water outlet of the water cooling device 5 respectively. When the confocal Raman spectra is collected, the semiconductor chilling plate 3 cools the bottom plate 4, and meanwhile, the cooling tube 2 is in communication with the water cooling device 5, such that the cooling tube 2 assists the semiconductor chilling plate 3 in cooling. The stainless steel slide is placed on the bottom plate 4, such that a temperature of the detection sample is reduced, and high temperature caused by a thermal effect of laser is overcome. A temperature difference after cooling does not partially condense water in air, such that the detection sample is moisturized, the detection sample is prevented from cracking, and an original shape of the detection sample is kept. That is, a signal-noise ratio, shape maintenance and protein stability of the detection sample are improved, and quality and reliability in a Raman spectrum collection process are improved.

Experimental results of signal-noise ratios of Raman spectra under conditions of using confocal Raman spectra cooling apparatus and conducting traditional normal-temperature collection under different air exposure time showed that SNRs of the Raman spectra decreased gradually both in normal brain tissue and glioma tissue under air exposure at room temperature, where the SNRs of the Raman spectra after exposure for 2 months were significantly lower than those after exposure for 2 minutes at room temperature and exposure for 2 hours at low temperature (P<0.05 in all cases), and the SNRs of the Raman spectra after exposure for 2 hours at low temperature were higher than those at room temperature and close to those after exposure for 2 minutes (Raman integral time: 10 S, A of FIG. 1). When glioma tissue at different Raman integral time was compared, a Raman SNR at room temperature for 10 S was higher than that at room temperature for 8 S, and a Raman SNR at low temperature for 8 S after cooling was significantly higher than that at room temperature (air exposure time: 2 hours, B of FIG. 1). It was indicated that for both normal brain tissue or glioma tissue, low-temperature Raman collection after cooling can resist SNR reduction caused by increase in time, and meanwhile, the shorter Raman collection integral time can be used to obtain the higher Raman SNR, which saved Raman collection time.

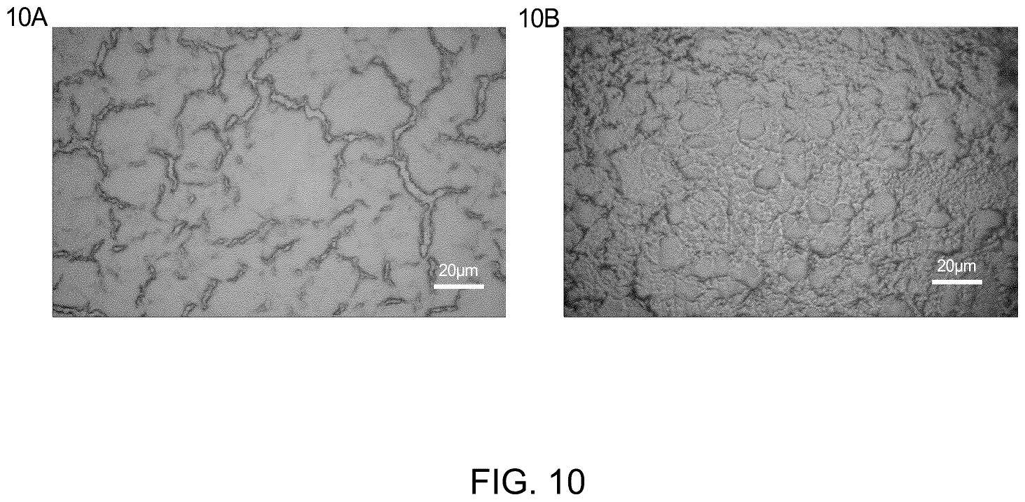

In order to further verify influence of the cooling apparatus on the shape of the detection sample, a comparative experiment was conducted. Ice-cut glioma samples were collected with and without the cooling apparatus. As shown in A of FIG. 10, ice-cut glioma tissue collected without the cooling apparatus was cracked and poor in shape, while ice-cut glioma tissue collected with the cooling apparatus was not cracked and good in shape.

What is claimed is:

1. A molecular imaging method of Raman spectra based on machine learning cascade, comprising the following steps:
(1) attaching an untreated frozen tissue slice to a stainless steel slide such that a detection sample is obtained, and then attaching an adjacent tissue slice to a glass slide such that a control sample is obtained;
(2) independently packaging the detection sample, storing the detection sample at 20° C. or below, conducting immunohistochemistry (IHC) staining on the control sample, obtaining an IHC image, selecting and defining a region of interest (ROI) on the IHC image, placing the stainless steel slide to which the detection sample is attached in a confocal Raman white-light field, obtaining a Raman white light image, and collecting Raman spectra of the ROI corresponding to a position of the IHC image in the Raman white light image;
(3) inputting the collected Raman spectra into a hierarchical clustering analysis module, obtaining Raman spectra of different types of biomolecules in the ROI, excluding other types of Raman spectra according to characteristic peaks of different types of Raman spectra, and reserving pure Raman spectra of a target biomolecule in the ROI;
(4) respectively inputting different types of obtained Raman spectra in different ROIs into a plurality of machine learning method models for training, obtaining a plurality of machine learning classification models, evaluating the plurality of machine learning classification models, selecting a machine learning classification model having optimal performance for creation of different types of Raman prediction models as a final Raman predictive imaging model, and obtaining a Raman predictive image and a quantitative score of a target biomolecule of the Raman predictive image;
(5) evaluating similarity between the IHC image and the Raman predictive image predicted through the Raman predictive imaging model with a similarity analysis module, and evaluating correlation between quantitative scores of target biomolecules of the IHC image and the Raman predictive image, that is, evaluating reliability of the Raman predictive image of the final Raman predictive imaging model; and
(6) preprocessing Raman spectra collected at any position of a sample to be detected, then inputting the preprocessed Raman spectra into the Raman predictive imaging model, and obtaining a Raman image and a quantitative score of a target biomolecule.

2. The molecular imaging method of Raman spectra based on machine learning cascade according to claim 1, wherein when the Raman white light image is obtained, the stainless steel slide to which the detection sample is attached is placed on a cooling apparatus, the cooling apparatus is arranged on an objective table of confocal Raman spectra, the cooling apparatus comprises a base, a cooling tube arranged on the base, a semiconductor chilling plate arranged on the cooling tube, and a bottom plate configured to bear stainless steel and glass slides, and two ends of the cooling tube are in communication with a pipe of a water cooling device.

3. The molecular imaging method of Raman spectra based on machine learning cascade according to claim 1, wherein selecting and defining the ROI on the IHC image comprises the following specific steps:
selecting an anatomical marker point on the IHC image, and coloring the anatomical marker point as a reference point;
defining the ROI around the reference point;
reserving a scale bar and a numerical value of an image of the ROI, reserving the image of the ROI, saving the image as an image file, converting the image file into a binary image, and removing pixels exceeding a threshold;
retrieving a contour in the binary image through a findContours function, and obtaining a vertex position of the ROI with a contour index; and
locating the reference point in the binary image at an origin (0,0), and establishing a two-dimensional coordinate system at the origin, wherein
computation formulas of vertex coordinates of a bounding box of the ROI are as follows:
$${x}_{d} = \frac{{x}_{v} - {x}_{p}}{len ( ruler )} \times scale \\ {y}_{d} = \frac{{y}_{v} - {y}_{p}}{len ( ruler )} \times scale$$
wherein xᵥ, yᵥ, xₚ, and yₚ denote positions of a vertex v and an origin p of the binary image, respectively, scale denotes the scale bar, len(ruler) denotes a length of the scale bar, and x_{d} and y_{d} denote scaling coordinates of the vertex.

4. The molecular imaging method of Raman spectra based on machine learning cascade according to claim 1, wherein collecting the Raman spectra of the ROI corresponding to the position of the IHC image in the Raman white light image comprises the following specific steps:
adjusting the IHC image and the Raman white light image, and making the IHC image and the Raman white light image at the same angle; and
keeping the Raman white light image and the IHC image the same in magnification ratio, selecting an origin and ROI vertexes at the same position as the IHC image on the Raman white light image, and collecting the Raman spectra of a corresponding ROI on the Raman white light image.

5. The molecular imaging method of Raman spectra based on machine learning cascade according to claim 1, wherein before the Raman spectra of the ROI are inputted into the hierarchical clustering analysis module, standard Raman spectra of different types of cells or standard proteins are collected, and Raman characteristic peaks of different types of biomolecules are obtained; and before the Raman spectra of the ROI are inputted into the hierarchical clustering analysis module, the Raman spectra of the ROI are preprocessed.

6. The molecular imaging method of Raman spectra based on machine learning cascade according to claim 1, wherein machine learning methods comprise support vector machine, random forest, linear discriminant analysis, gradient boosting trees, and deep learning; and
evaluating the plurality of machine learning classification models comprises generating a plurality of types of receiver operating characteristic curves and using an area under the plurality of types of receiver operating characteristic curves as an evaluation index while evaluating performance of the machine learning classification model with mean sensitivity, specificity and accuracy.

7. The molecular imaging method of Raman spectra based on machine learning cascade according to claim 1, wherein according to staining colors of different target biomarkers in the IHC image, a prediction result of the Raman predictive imaging model is given a corresponding pseudo-color; and frequency of each predicted value of machine learning classification model is computed through a table function, and then a ratio of the number of different types of Raman spectra to a total Raman spectrum number is obtained through prop according to a table function.

8. The molecular imaging method of Raman spectra based on machine learning cascade according to claim 1, wherein evaluating the reliability of the Raman predictive image of the Raman predictive imaging model comprises the following steps:
selecting the ROI from the IHC image, obtaining coordinate values of the ROI, and obtaining the Raman spectra of a corresponding ROI in the Raman white light image according to the coordinate values; and
inputting the collected Raman spectra and Raman predictive image into the similarity analysis module, and evaluating brightness, contrast and structural similarity between the Raman predictive image and the IHC image of the adjacent slice, wherein
$$SSIM = {[ l ( x , y ) ]}^{α} \cdot {[ c ( x , y ) ]}^{β} \cdot {[ s ( x , y ) ]}^{γ} \\ l ( x , y ) = \frac{2 {μ}_{x} {μ}_{x} + {C}_{1}}{{μ}_{x}^{2} + {μ}_{y}^{2} + {C}_{1}} \\ c ( x , x ) = \frac{2 {σ}_{x} {σ}_{y} + {C}_{2}}{{σ}_{x}^{2} + {σ}_{y}^{2} + {C}_{2}} \\ c ( x , y ) = \frac{{σ}_{xy} + {C}_{3}}{{σ}_{x} {σ}_{y} + {C}_{3}}$$
x denotes the IHC image, y denotes the Raman predictive image, l(x,y), c(x,y), and s(x,y) denote brightness comparison, contrast comparison and structure comparison, respectively, μₓ, μ_{y}, σₓ and σ_{y} denote mean intensities and standard deviations of x and y, respectively, C₁, C₂, and C₃ denote constant terms, an exponential condition is set as “α=β=γ=1”, and in consideration that computation of structural similarity (SSIM) is based on a single-color region of the IHC image or the Raman predictive image, a color region is separated through k-means.

9. A molecular imaging system of Raman spectra based on machine learning cascade, comprising:
a coordinate localization module configured to obtain coordinates of ROIs of an IHC image and a Raman white light image;
a hierarchical clustering analysis module configured to conduct classification and purification on the Raman spectra in the ROI and obtain the Raman spectra of a target biomolecule in the ROI;
a Raman predictive imaging module configured to predict a molecular type of a sample to be detected and build a Raman image, and to obtain a Raman predictive image and a quantitative score of a target biomolecule of the Raman predictive image; and
a similarity analysis module configured to evaluate similarity between the Raman predictive image of the Raman predictive imaging module and the IHC image, and to evaluate correlation between quantitative scores of target biomolecules of the Raman predictive image and the IHC image.

10. The molecular imaging system of Raman spectra based on machine learning cascade according to claim 9, wherein according to the coordinate localization module, a stainless steel slide is used as a substrate, an untreated frozen tissue slice is attached to the stainless steel slide and kept at 20° C. or below, then an adjacent tissue slice is attached to a glass slide, the two slices are kept at the same angle, IHC staining is conducted on the tissue slice on the glass slide, the IHC image is obtained, an anatomical marker point is selected on the IHC image and colored as a reference point, a ROI is defined around the reference point, a scale bar and a numerical value of an image of the ROI are reserved, the image of the ROI is reserved, the image is saved as an image file, the image file is converted into a binary image, pixels exceeding a threshold are removed, a contour is retrieved in the binary image through a findContours function, a vertex position of the ROI is obtained with a contour index, the reference point in the binary image is located at an origin (0,0), a two-dimensional coordinate system is established at the origin, and computation formulas of vertex coordinates of a bounding box of the ROI are as follows:
$${x}_{d} = \frac{{x}_{v} - {x}_{p}}{len ( ruler )} \times scale \\ {y}_{d} = \frac{{y}_{v} - {y}_{p}}{len ( ruler )} \times scale$$
wherein xᵥ, yᵥ, xₚ, and yₚ denote positions of a vertex v and an origin p of the binary image, respectively, scale denotes the scale bar, len(ruler) denotes a length of the scale bar, and x_{d} and y_{d} denote scaling coordinates of the vertex; and a detection sample attached to the stainless steel slide is placed in a confocal Raman white-light field, the Raman white light image is obtained, the Raman white light image and the IHC image are kept to be the same in magnification ratio, an origin and ROI vertexes at the same position as the IHC image are selected on the Raman white light image, and the Raman spectra of a corresponding ROI are collected on the Raman white light image;
according to the hierarchical clustering analysis module, the Raman spectra of other types of biomolecules in the ROI are excluded with the hierarchical clustering analysis module, different types of the Raman spectra are obtained, the other types of the Raman spectra are excluded according to characteristic peaks of the different types of Raman spectra, and pure Raman spectra of a target biomolecule in the ROI are reserved;
according to the Raman predictive imaging module, different types of Raman spectra are firstly predicted with different machine learning method models respectively, a machine learning classification model having optimal performance is selected for creation of Raman prediction models of different types of biomolecules as a final Raman predictive imaging model, then according to staining colors of different target biomolecule markers in the IHC image, a prediction result of the Raman predictive imaging model is given a corresponding pseudo-color, the Raman predictive image is obtained, and proportional scores of the different types of biomolecules are computed according to proportions of different types;
according to the similarity analysis module, the ROI is selected from the IHC image, coordinate values of the ROI are obtained, Raman spectra of a corresponding ROI in the Raman white light image are obtained according to the coordinate values, the collected Raman spectra are preprocessed and then inputted into the Raman predictive imaging model, the Raman predictive image is obtained, the Raman predictive image and an IHC image of an adjacent slice are inputted into the similarity analysis module, and brightness, contrast and structural similarity between the Raman predictive image and the IHC image of the adjacent slice are evaluated,
$$SSIM = {[ l ( x , y ) ]}^{α} \cdot {[ c ( x , y ) ]}^{β} \cdot {[ s ( x , y ) ]}^{γ} \\ l ( x , y ) = \frac{2 {μ}_{x} {μ}_{x} + {C}_{1}}{{μ}_{x}^{2} + {μ}_{y}^{2} + {C}_{1}} \\ c ( x , x ) = \frac{2 {σ}_{x} {σ}_{y} + {C}_{2}}{{σ}_{x}^{2} + {σ}_{y}^{2} + {C}_{2}} \\ c ( x , y ) = \frac{{σ}_{xy} + {C}_{3}}{{σ}_{x} {σ}_{y} + {C}_{3}}$$
x denotes the IHC image, y denotes the Raman predictive image, l(x,y), c(x,y), and s(x,y) denote brightness comparison, contrast comparison and structure comparison, respectively, μₓ, μ_{y}, σₓ and σ_{y} denote mean intensities and standard deviations of x and y, respectively, C₁, C₂, and C₃ denote constant terms, an exponential condition is set as “α=β=γ=1”, and in consideration that computation of SSIM is based on a single-color region of the IHC image or the Raman predictive image, a color region is separated through k-means; and
data obtained by preprocessing the Raman spectra collected at any position of the tissue slice of the sample to be detected is inputted into the Raman predictive imaging module, such that the Raman image and a quantitative score of a target molecule are obtained.
